(19) United States
(12) Patent Application Publication
(10) Pub. No.: US 2024/0033207 A1
(43) Pub. Date: Feb. 1, 2024

(54) TOPICALLY ADMINISTRABLE LYSATE (LY) OF DEDIFFERENTIATED CELLS OF THE PLANT HELICHRYSUM STOECHAS FOR ELIMINATING OR REDUCING INFLAMMATION OF THE SKIN

(71) Applicants: SOCIETE D'EXPLOITATION DE PRODUITS POUR LES INDUSTRIES CHIMIQUES - SEPPIC, PARIS CEDEX 07 (FR); BIOTECHMARINE, Quemper Guezennec (FR)

(72) Inventors: Erwan LE GELEBART, QUEMPER-GUÉZENNEC (FR); Cécile BIZE, CASTRES (FR)

(21) Appl. No.: 17/800,187

(22) PCT Filed: Feb. 15, 2021
(86) PCT No.: PCT/EP2021/053648

§ 371 Date: Aug. 16, 2022

(30) Foreign Application Priority Data
Feb. 17, 2020 (FR) 2001548

(51) Int. Cl. A61K 8/9789; A61Q 19/00; A61K 8/06

(52) U.S. Cl. CPC A61K 8/9789; A61Q 19/005; A61K 8/062; A61K 2236/37

(57) ABSTRACT

Disclosed is a lysate of dedifferentiated cells of the plant Helichrysum stoechas, which can be topically administered, for eliminating or reducing inflammation of the skin and/or scalp.

### CROSS-REFERENCE TO RELATED APPLICATIONS

This application is the U.S. national phase of International Application No. PCT/EP2021/053648 filed Feb. 15, 2021 which designated the U.S. and claims priority to French Patent Application No. 2001548 filed Feb. 17, 2020, the entire contents of each of which are hereby incorporated by reference.

### BACKGROUND OF THE INVENTION

### Field of the Invention

15 The present invention relates to a topically administrable lysate of dedifferentiated cells of the plant Helichrysum stoechas for soothing human skin and/or the scalp, more particularly dry, reactive and/or sensitive skin.

### Description of the Related Art

The skin constitutes the interface between the human internal medium (or human body) and the external environment. As a result, and with the flora which covers and inhabits it, the skin has in particular the function of performing a mission of protection of the human body, by forming a genuine barrier which is vital. In particular, the skin makes it possible to combat dehydration by limiting the diffusion of water out of the body.

Due to its interface position with the external environment, the skin is subjected to numerous daily stresses, such as, for example, contact with clothing, changes in temperature, exposure to the ultraviolet radiation of sunlight, changes in humidity levels, contact with certain irritating chemicals or contact with chemicals regarded as polluting agents.

The skin is composed of layers of different tissues:
the epidermis, composed of keratinocytes, is its outermost part, followed by
the dermis, which is a connective tissue mainly composed of fibroblasts and of extracellular matrix, and
the hypodermis, consisting of adipocytes, which is the deepest part and the part furthest from the external environment.

The skin performs various functions in the interest of the entire system which it shelters, among which are included the following:
a mechanical barrier function in order to guarantee the integrity of the internal medium of the body,
an emunctorial function directed at secreting sweat based on water, on salts and on acidic waste,
a function of regulating the temperature of the body, and contains many other regulatory mechanisms, such as, for example, its mechanism of adaptation and of protection against ultraviolet radiation (adaptive pigment coloring by the production of melanin), such as, for example, a system for immune monitoring by the presence of macrophages or dendritic cells.

The human skin also constitutes the first image offered to the eyes of others. Consequently, improving its appearance is a subject of constant concern to human beings. The skin is the reflection of a state of well-being, often associated with youthfulness, and, conversely, with a state of tiredness and/or aging. The result of this is that preserving and improving the state of the outermost layer of the skin, namely the epidermis, is a major center of interest for the research studies carried out by the cosmetics industries.

At the periphery of the epidermis is an upper horny layer known as the stratum corneum, which is the first layer of the epidermis to be subjected to stresses of external origin, such as variations in external weather conditions (temperature, hygrometry, ultraviolet radiation, polluting agents) or mechanical stresses. As regards the soothing of reactive and/or sensitive skin, sensitive or reactive skin is a condition which affects many people: approximately 50% of people (60% of women, 40% of men) report having reactive skin.

Sensitive skin is defined by a specific reactivity of the skin.

This skin reactivity is expressed conventionally by the manifestation of signs of discomfort, such as red patches, in response to the subject being placed in contact with a triggering element, which may have various origins.

It was recently defined as follows: “a syndrome defined by the occurrence of unpleasant sensations (stinging, burning, pain, pruritus and tingling sensations) in response to stimuli that normally should not provoke such sensations. These sensations cannot be explained by lesions attributable to any skin disease. The skin can appear normal or be accompanied by erythema. Sensitive skin can affect all parts of the body, especially the face”.

Environmental factors, such as exposure to ultraviolet or infrared rays, and/or to atmospheric pollution, and/or to sudden variations in temperature and/or to the wind, or lifestyle (food habits or application of cosmetic products at the surface of the skin), or also physiological factors, such as stress, endogenous hormones, have been recognized as being able to induce or aggravate the symptoms of sensitive skin. Two main reasons can explain the symptoms of sensitive skin: on the one hand, an increase in the permeability of the stratum corneum and, on the other hand, excessive secretion of certain neurotransmitters by the endings of the superficial nerves and proinflammatory cytokines.

This is because sensitive skin undergoes an inflammatory cascade. The excessive production of proinflammatory cytokines maintains the inflammation of sensitive skin and the associated visible manifestations (red patches, tightness, itching, uncomfortable sensations of heat).

Within the meaning of the present invention, sensitive skin embraces irritable skin and intolerant skin.

Intolerant skin is skin which reacts via sensations of heating, tightness, tingling and/or red patches to various factors, such as the application of cosmetic or dermatological products or of soap. Generally speaking, these signs are associated with erythema and with hyperseborrheic or acneic skin, or even rosaceiform skin, with or without dry patches.

Irritable skin is skin which reacts by a pruritis, that is to say by itching or by stinging, to various factors, such as the environment, emotions, foods, wind, rubbing actions, shaving, hard water having a high calcium concentration, temperature variations, humidity or wool.

From there, a problem which arises is that of providing a compound making it possible to soothe the skin, to reduce its inflammation.

### SUMMARY OF THE INVENTION

A solution according to the present invention is a topically administrable lysate (Ly) of dedifferentiated cells of the plant Helichrysum stoechas for eliminating or reducing inflammation of the skin and/or of the scalp.

The term “eliminating or reducing inflammation of the human skin and/or of the scalp” is understood to mean, within the meaning of the present invention, the partial or complete return to the state of homeostasis of the inflamed cells.

The term “topically administrable” is understood to mean that the lysate is formulated in order to be topically administrable.

The lysate (Ly) of dedifferentiated cells of the plant Helichrysum stoechas will be the active principle which makes it possible to eliminate the inflammation of the skin and/or scalp.

According to a specific aspect, the present invention relates to a topically administrable lysate of dedifferentiated cells of the plant Helichrysum stoechas for eliminating the unsightly and/or symptomatic signs linked to the inflammation of human skin and/or of the scalp, such as, for example, red patches, tightness, itching or stinging.

Preferably, the lysate results from the high-pressure homogenization of the culture of dedifferentiated cells of the plant Helichrysum stoechas.

The process for obtaining the lysate of dedifferentiated cells of the plant Helichrysum stoechas according to the invention will preferably comprise the following stages:
a) Preparation of the sterile sample of the plant Helichrysum stoechas
b) Callogenesis
c) Suspending
d) Selection of a fine suspension
e) Biomass production
f) High-pressure homogenization of the culture
g) Stabilization of the lysate of culture of dedifferentiated cells.

From stage e), stages a), b), c) and d) no longer need to be repeated because it leads to the culture of dedifferentiated cells which can be used ad infinitum to produce biomass insofar as the physiological needs of the cells are respected.

Stages b), c), d) and e) must be carried out under sterile conditions in order to maintain the culture under axenic conditions.

The stages are described in detail as follows:

a) Preparation of the Sterile Sample of Helichrysum stoechas

A few leaves of Helichrysum stoechas are removed, and these leaves are cut into fragments of a few millimeters. The fragments of leaves are sterilized by immersion in successive baths containing:
a mixture of 70% ethanol containing a detergent, such as Tween 80, in order to improve the effectiveness of the sterilization for a period of time of 5 minutes, then
a 1% by weight sodium hypochlorite solution for a period of time of 5 minutes The fragments of the leaves thus sterilized are then rinsed by immersion in a bath of sterile distilled water 3 times in succession.

This stage a) can also be carried out using any other part of the plant (roots, stems, meristems, flowers, and the like). The sterilization can also be applied to the seeds, without the cutting phase.

b) Callogenesis

Pieces of sterilized leaves obtained in the preceding stage are placed on the solid callogenesis medium (the composition of which is described in table 1), which is subsequently incubated for a period of time of three weeks in a thermostatically controlled chamber at a temperature of greater than or equal to 20° C. and less than or equal to 25° C. in the light or in the dark.

TABLE 1

| Compounds | Concentration (mg/l) |
|---|---|
| Glucose or Sucrose | 30000 |
| Naphthaleneacetic acid | 1 |
| 6-Benzylaminopurine | 0.5 |
| Agar | 10000 |
| KNO₃ | 2500 |
| (NH₄)₂SO₄ | 134 |
| CaCl₂•2H₂O | 150 |
| NaH₂PO₄•2H₂O | 150 |
| MgSO₄•7H₂O | 250 |
| MnSO₄•H₂O | 16.9 |
| ZnSO₄•7H₂O | 8.6 |
| H₃BO₃ | 6.2 |
| KI | 0.83 |
| Na₂MoO₄•2H₂O | 0.25 |
| CuSO₄•5H₂O | 0.025 |
| FeSO₄•7H₂O | 27.8 |
| Na₂EDTA•2H₂O | 37.3 |
| Myo-inositol | 100 |
| Nicotinic acid | 1 |
| Pantothenic acid | 1 |
| Biotin | 0.01 |
| Pyridoxine | 1 |
| Thiamine | 1 |

Composition of the Callogenesis Medium

c) Suspending the Dedifferentiated Cells

The callus formed during stage b) is withdrawn and placed in a liquid suspending medium (the composition of which is shown in table 2). The term “callus” is understood to mean, within the meaning of the present invention, a cluster of dedifferentiated cells.

The pieces of callus in suspension are cultured in a thermostatically controlled chamber for a period of time of 7 days to 21 days at a temperature of greater than or equal to 20° C. and of less than or equal to 25° C. in the light or in the dark, with mechanical stirring of orbital type, at a stirring speed of between 25 revolutions/minute and 200 revolutions/minute (more specifically at a stirring speed of 100 revolutions/minute for an orbital diameter of 5 cm).

TABLE 2

| Compounds | Concentration (mg/l) |
|---|---|
| Glucose or Sucrose | 30000 |
| Naphthaleneacetic acid | 1 |
| 6-Benzylaminopurine | 0.5 |
| KNO₃ | 2500 |
| (NH₄)₂SO₄ | 134 |
| CaC1₂.2H₂O | 150 |
| NaH₂PO₄.2H₂O | 150 |
| MgSO₄.7H₂O | 250 |
| MnSO₄.H₂O | 16.9 |
| ZnSO₄.7H₂O | 8.6 |
| H₃BO₃ | 6.2 |
| KI | 0.83 |
| Na₂MoO₄.2H₂O | 0.25 |
| CuSO₄.5H₂O | 0.025 |
| FeSO₄.7H₂O | 27.8 |
| Na₂EDTA.2H₂O | 37.3 |
| Myo-inositol | 100 |
| Nicotinic acid | 1 |
| Pantothenic acid | 1 |
| Biotin | 0.01 |
| Pyridoxine | 1 |
| Thiamine | 1 |

Composition of the medium for suspending the callus formed in stage b).

d) Selection of a Fine Suspension

Between 1/5 and 1/3 of the volume of culture containing dedifferentiated cells in fine suspension which are obtained on conclusion of stage c) is withdrawn.

This volume of fine suspension is placed in a volume of fresh suspending medium, three times greater than the volume of fine suspension.

This new suspension is cultured in a thermostatically controlled chamber fora period of time of 7 to 21 days (the end of the culturing period is dictated by the observation of the deficiency in nutrients) at a temperature of greater than or equal to 20° C. and of less than or equal to in the light, with mechanical stirring of orbital type, at a rotational speed of between 25 revolutions/minute and 200 revolutions/minute (more specifically at a rotational speed of 100 revolutions/minute for an orbital diameter of 5 cm). The term “deficiency in nutrients” is understood to mean, within the meaning of the present invention, the observation of a content of less than 5% of the amount of nutrient initially introduced into the culture medium.

e) Production of Biomass Comprising Dedifferentiated Cells and the Culture Medium

A volume of the fine suspension obtained during stage d) is withdrawn and deposited in a volume of fresh suspending medium, as described in table 2, which is 3 times greater than the withdrawn volume of fine suspension obtained on conclusion of stage d).

This new suspension is cultured in a thermostatically controlled chamber fora period of time of 7 to 21 days (the end of the culturing period is dictated by the deficiency in nutrients) at a temperature of greater than or equal to 20° C. and of less than or equal to 25° C., in the light or in the dark, with mechanical stirring of orbital type, at a rotational speed of between 25 revolutions/minute and 200 revolutions/minute (more specifically at 100 revolutions/minute for an orbital diameter of 5 cm). This stage can be carried out, up to volumes of less than or equal to 5 liters, in Erlenmeyer flasks, then, for volumes of greater than 5 liters and less than or equal to 1000 liters, in bioreactors (“wave reactor”, “stirred tank reactor” or Erlenmeyer flask type) (this list not being exhaustive).

f) High-Pressure Homogenization of the Culture: Obtaining the Lysate of Culture of Dedifferentiated Cells

A fraction of the volume of culture obtained on conclusion of stage e) is withdrawn to be lysed using a high-pressure homogenizer, the pressure of which can be set at a value of greater than or equal to 100 bars and less than or equal to 1000 bars (preferably, for the present invention, at a pressure equal to 500 bars). The dedifferentiated cells present in the culture passed through the high-pressure homogenizer are lysed, and their contents are released into the culture medium. The homogenization efficiency (which is expressed by the observation of cell debris testifying to the lysis of the dedifferentiated cells) can be easily verified by microscopic observation of the suspension.

g) Stabilization of the Lysate of Culture of Dedifferentiated Cells

The lysate of culture of dedifferentiated cells which is collected on conclusion of stage f) is acidified, by the addition of an inorganic acid or of an organic acid, in order to achieve a pH value of greater than or equal to 4 and of less than or equal to 4.5, in order to stabilize its color. This lysate contains pieces of cells which are insoluble in the medium and which are liable to settle out, and to constitute an obstacle to the commercialization of the product. The addition of a thickening and/or gelling agent, in a proportion of greater than or equal to 0.1% by weight and of less than or equal to 2% (more preferentially a mixture of xanthan gum and acacia gum, in proportions by weight of 40-45% and 48-55% and in an amount of 0.8% by weight, such as the mixture of xanthan gum and of acacia gum sold under the brand name Solagum™ AX), makes it possible to obtain a suspension which does not settle out over time.

A lysate of dedifferentiated cells can be stabilized from a microbiological and physicochemical viewpoint by various means:
by pasteurization (example: UHT pasteurization),
by modification of the pH (example: acidification pH<5),
by addition of an agent for reducing the availability of water (example: glycerol),
by addition of preservatives (example: sodium benzoate, potassium sorbate).

In our case, we prefer to stabilize our lysate by the addition of a mixture of glycerol, potassium sorbate and sodium benzoate.

The proportions of the various ingredients are as follows, per 100% by weight:
60% to 70% by mass of HPH lysate from the culture of dedifferentiated cells of Helichrysum stoechas, obtained in stage f) (95% to 99.5% by weight of water and 0.5% to 5% by weight of dry matter)
30% to 40% by weight of glycerol
0.1% to 2% by weight of Solagum™ AX (mixture of acacia gum and xanthan gum)
0.1% to 2% of potassium sorbate
0.1% to 2% of sodium benzoate.

The stabilized lysate of culture of dedifferentiated cells can be stored or be directly incorporated in a cosmetic formulation in an amount of 0.1% to 3% by weight (preferentially 1% by weight).

Any kind of thickening agent can perform the function of stabilizing agent; for example we use a solvent of a polar nature, preferably glycerol, but other polyols might be used. It should be noted that there are many differences between the whole plant and the dedifferentiated cells. To show these differences, we carried out a comparison. The comparison between the whole plant and the dedifferentiated cells could be carried out by virtue of the comparison of ethanolic extracts analyzed by high pressure liquid chromatography (or HPLC). The samples are analyzed on a Kinetex C18 150*4.6 mm HPLC column with a flow rate of 0.8 ml/min. The solvents are as follows:
A: isopropanol having 0.05% TFA,
B: acetonitrile having 0.05% TFA,
C: water having 0.05% TFA.

The gradient is as follows:

| t (min) | % A | % B | % C |
|---|---|---|---|
| 0 | 0 | 2 | 98 |
| 5 | 0 | 2 | 98 |
| 20 | 0 | 100 | 0 |
| 25 | 0 | 100 | 0 |
| 35 | 80 | 20 | 0 |
| 40 | 80 | 20 | 0 |
| 45 | 0 | 100 | 0 |
| 50 | 0 | 2 | 98 |

This analysis was carried out with detection of CAD (Charged Aerosol Detection) type. The results are obtained from a chromatogram of an ethanolic extract of aerial parts of Helichrysum stoechas in CAD detection according to the HPLC C18_screening method and a chromatogram of an ethanolic extract of dedifferentiated cells of Helichrysum stoechas in CAD detection according to the HPLC C18_screening method.

The results obtained clearly show a difference between the two chromatograms. The chromatogram of the cell extract is much richer in the region between the retention times of between 25 minutes and 40 minutes, which corresponds to the elution times of the most nonpolar molecules.

Another subject matter of the present invention is a composition for topical use (C1) which is provided in the form of a gel and which comprises, per 100% of its weight:
from 95% to 99.5% by weight, preferably from 97% to 99.5% by weight, more preferentially still from 97.5% to 99.5% by weight, of the lysate (Ly) as defined above and preferably obtained by the implementation of a process comprising stages a) to f) as described above,
from 0.5% to 5% by weight, preferably from 0.5% to 3% by weight, more preferentially still from 0.5% to 2.5% by weight, of at least one thickening and/or gelling agent.

Preferably, the gelling and/or thickening agents are chosen from polysaccharides, cellulose and cellulose derivatives, starches and linear or branched or crosslinked polymers of polyelectrolyte type.

This composition (C1) can be topically administrable for eliminating or reducing inflammation of the skin and/or of the scalp. The term “topically administrable” is understood to mean that the composition is formulated in order to be topically administrable.

According to an even more specific aspect, the present invention relates to a topically administrable composition (C1) for eliminating or reducing the unsightly and/or symptomatic signs linked to the inflammation of sensitive human skin and/or of the scalp, such as, for example, red patches, tightness, itching or stinging.

In the context of the present invention, the term “gel” denotes a chemical composition which is provided initially in the liquid state and which, after addition of gelling and/or thickening substances, is transformed into a structured state, which does not flow. A gel such as defined is regarded as an intermediate state between the solid state and the liquid state, and consists of a three-dimensional network within the liquid which is the result of chemical or physical bonds.

Within the meaning of the present invention, the term “gelling agent” is understood to mean a chemical compound or a mixture of chemical compounds which transforms a liquid medium into a gel.

Within the meaning of the present invention, the term “thickening agent” is understood to mean a chemical compound or a mixture of chemical compounds which increases the viscosity of the medium into which it is introduced.

The expression “for topical use” used in the definition of the composition (C₁) as described above means that said composition will be formulated in order to make possible its application to the skin, hair, scalp, nails, lips, mucus membranes, eyelashes or eyebrows, whether a direct application, in the case of a cosmetic formulation, or an indirect application, for example in the case of a body care product in the form of a textile or paper wipe or of sanitary products intended to be in contact with skin, hair, scalp, nails, lips, mucus membranes, eyelashes or eyebrows.

In the context of the present invention, the term “polysaccharides” denotes saccharide polymers. The IUPAC definition of saccharides designates monosaccharides, compounds of monosaccharides proper and their derivatives, obtained either by reduction of a carbonyl group, or by oxidation of one or more hydroxyl functions, or by the replacement of one or more hydroxyl functions by a hydrogen atom, an amine group, a phosphate function or a sulfate function. The polysaccharides most commonly used for the preparation of industrial, food, cosmetic or pharmaceutical compositions predominantly consist of monosaccharides, such as glucose, galactose or mannose, or of monosaccharide derivatives for which the hydroxyl function of the terminal carbon has been oxidized to give a carboxyl function. Two distinct groups may be distinguished among the polysaccharides: polysaccharides consisting solely of monosaccharides (or poly-monosaccharides) and polysaccharides consisting of monosaccharide derivatives.

According to a specific aspect, the gelling and/or thickening agents present in the aqueous composition (C1) which is a subject matter of the present invention are chosen from polysaccharides consisting solely of monosaccharides (or poly-monosaccharides). Among the polysaccharides composed solely of monosaccharides, a distinction may be made between glucans, which are glucose homopolymers very abundant in nature, glucomannoglycans, xyloglycans and galactomannans, which are polymers, the main chain of which consists of D-mannose units, connected together as β-1,4, and to which D-galactose units are grafted laterally by α-1,6 bonds.

Galactomannans are present in several plant species, and more particularly in the leguminous species in which they constitute the albumen of the seeds. Depending on their plant origin, the degree of substitution (DS) of the D-galactose units on the D-mannose main chain of galactomannans ranges between 0 and 1:
galactomannans originating from cassia gum exhibit a degree of substitution (DS) of approximately 1/5, meaning the lateral grafting of one D-galactose unit every 5 D-mannose units present on the main chain of the polysaccharide;
galactomannans originating from locust bean gum exhibit a degree of substitution (DS) of approximately 1/4, meaning the lateral grafting of one D-galactose unit every 4 D-mannose units present on the main chain of the polysaccharide;
galactomannans originating from tara gum exhibit a degree of substitution (DS) of approximately 1/3, meaning the lateral grafting of one D-galactose unit every 3 D-mannose units present on the main chain of the polysaccharide;
galactomannans originating from guar gum exhibit a degree of substitution (DS) of approximately 1/2, meaning the lateral grafting of one D-galactose unit every 2 D-mannose units present on the main chain of the polysaccharide;
galactomannans originating from fenugreek gum exhibit a degree of substitution (DS) of approximately 1/1, meaning the lateral grafting of one D-galactose unit for virtually every D-mannose unit present on the main chain of the polysaccharide.

According to a more specific aspect, the gelling and/or thickening agents present in the aqueous composition (C1) which is a subject matter of the present invention are chosen from polysaccharides consisting solely of monosaccharides (or poly-monosaccharides) included in the group consisting of galactomannan originating from tara gum, galactomannan originating from guar gum and galactomannan originating from locust bean gum.

According to another specific aspect, the gelling and/or thickening agents present in the aqueous composition (C1) which is a subject matter of the present invention are chosen from polysaccharides consisting of monosaccharide derivatives. Among the polysaccharides consisting of monosaccharide derivatives, a distinction may be made between:
sulfated galactans, which are galactose polymers which may have pendant sulfate-ester groups, represented in particular by algal polysaccharides, such as carrageenans and agar;
uronans, which are the polymers of uronic acids, such as algins and pectins;
heteropolymers of monosaccharides and uronic acids: often of complex composition, these polymers are found in particular in sap exudates (such as, for example, gum arabic exudate and karaya gum exudate) but they are also produced by microorganisms, such as, for example, xanthan gum and gellan gum;
glucosaminoglycans, which are polysaccharides formed from a glucose derived by replacement of its C-2 hydroxyl by an amine (referred to as 2-amino-2-deoxy-D-glucose or, more simply, glucosamine). The amine function can furthermore be acetylated. The hydrocolloids in this class include chitosan, formed solely of glucosamine units, and hyaluronan, the repeat unit of which is a dimer of glucosamine and of glucuronic acid.

Xanthan gum (Gₓ) has in recent decades become the microbial polysaccharide most widely used in industry. Xanthan is a polysaccharide synthesized by bacteria of the genus Xanthomonas and, commercially, only the species X. campestris is used. The main chain of (Gₓ) is identical to that of cellulose, that is to say that it is formed of β-D-glucose units connected via the carbons 1 and 4. There is one branched trisaccharide every two glucose units in the main chain, in a regular alternating manner; each branch consisting of a trisaccharide composed of two mannoses and one glucuronic acid, of the type: β-D-Manp-(1→4)-B-D-GlcAp-(1→2)-A-D-Manp-(1→3) (I. Capron et al., “About the Native and Renaturated Conformation of Xanthan Exopolysaccharide”, 1997). Xanthan gum (Gₓ) is available in the form of a sodium, potassium or calcium salt. Acacia gum is a complex branched polysaccharide, the main chain of which consists of β-D-galactose units connected together via the carbons 1 and 3. The chains branched to the main chain consist of β-D-galactose units connected together via the carbons 1 and 6, also carrying α-arabinose units and, in lower proportions, β-glucoronosyl units. Both the main chain and the pendant chains contain α-L-arabinosyl, α-L-rhannnopyranosyl, β-D-glucuronopyranosyl and 4-O-methyl-β-D-glucuronpyranosyl units.

According to a more specific aspect, the gelling and/or thickening agents present in the aqueous composition (C₁) which is a subject matter of the present invention are polysaccharides consisting of monosaccharide derivatives chosen from the elements of the group consisting of carrageenans, agar, algins, pectins, gum arabic exudate, karaya gum exudate, xanthan gum, gellan gum, chitosan and hyaluronan, and/or their mixtures.

According to an even more specific aspect, the gelling and/or thickening agents present in the aqueous composition (C₁) which is a subject matter of the present invention are polysaccharides consisting of monosaccharide derivatives chosen from the elements of the group consisting of gum arabic exudate, karaya gum exudate and xanthan gum, and/or their mixtures.

According to an even more specific aspect, the gelling and/or thickening agents present in the aqueous composition (C₁) which is a subject matter of the present invention are polysaccharides consisting of monosaccharide derivatives chosen from the elements of the group consisting of gum arabic exudate, xanthan gum and the mixture of xanthan gum (Gₓ) and of gum arabic exudate (G_{A}) used in a ratio by weight of xanthan gum (Gₓ) to acacia gum exudate (G_{A}) of greater than or equal to 1/3 and less than or equal to 3/1, in particular sold by SEPPIC under the brand name Solagum™ AX.

According to a specific aspect, the gelling and/or thickening agents present in the aqueous composition (C₁) which is a subject matter of the present invention are chosen from cellulose and cellulose derivatives.

In the context of the present invention, the term “cellulose” denotes a polysaccharide consisting of a linear chain of D-glucose molecules, the average molecular weight of which is at least 10 000 g·mol⁻¹, more particularly at least 15 000 g·mol⁻¹, more particularly at least 17 000 g·mol⁻¹, more particularly still at least 20 000 g·mol⁻¹ and more particularly still at least 25 000 g·mol⁻¹.

According to a more specific aspect, the gelling and/or thickening agents present in the aqueous composition (C₁) which is a subject matter of the present invention are chosen from cellulose derivatives.

In the context of the present invention, the term “cellulose derivatives” denotes the elements of the group consisting of hydroxyethyl cellulose, methyl cellulose, ethyl cellulose, methyl hydroxyethyl cellulose, methyl hydroxypropyl cellulose, hydroxypropyl cellulose, the sodium salt of carboxymethyl cellulose and cellulose dihydroxypropyl ether.

In the context of the present invention, the term “starch” denotes a mixture of amylose and amylopectin, and more particularly the elements of the group consisting of corn starch, wheat starch, potato starch and cassava starch.

According to a specific aspect, the term “linear or branched or crosslinked polymers of polyelectrolyte type” denotes, within the meaning of the present invention:
crosslinked synthetic anionic copolymers based on methacrylic acid or on acrylic acid, or on esters of methacrylic acid or of acrylic acid, which are optionally hydrophically modified, prepared by direct emulsion polymerization. These synthetic anionic copolymers are respectively known to a person skilled in the art under the names “Alkaline Swellable Emulsion” (or “ASE”) and “Hydrophobically Alkaline Swellable Emulsion” (or “HASE”). Thickening agents of HASE type are described in the international patent application published under the number WO 02/34793 A2;
crosslinked or branched synthetic anionic polyelectrolytes, which are crosslinked and/or branched homopolymers or copolymers of water-soluble unsaturated monomers, such as acrylic acid and/or its derivatives, methacrylic acid and/or its derivatives, acrylamide and/or its derivatives, 2-acrylamido-2-methylpropanesulfonic acid and/or its salts, N-vinylpyrrolidone, vinyl alcohol and/or its derivatives. These crosslinked or branched synthetic anionic polyelectrolytes are provided in the form of inverse latexes, obtained by inverse emulsion radical polymerization, or in the form of powders, obtained by precipitating polymerization or by atomization of inverse latexes.

According to a specific aspect, the gelling and/or thickening agents present in the aqueous composition (C₁) which is a subject matter of the present invention are chosen from linear or branched or crosslinked polyelectrolytes, resulting from the radical polymerization of at least one monomer selected from the elements of the group consisting of acrylic acid and/or its sodium salt, methacrylic acid and/or its sodium salt, 2-hydroxyethyl acrylate, 2-hydroxyethyl methacrylate, acrylamide, N,N-dimethylacrylamide, N-isopropylacrylamide, 2-acrylamido-2-methylpropanesulfonic acid and/or its sodium or potassium salt, N-vinylpyrrolidone and at least one monomer of formula (I):

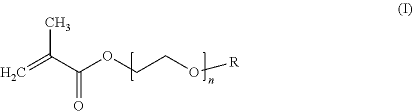

in which R represents a linear or branched alkyl radical comprising from 8 to 20 carbon atoms and n represents an integer of greater than or equal to 0 and less than or equal to 20. Said radical polymerization is carried out in the presence of a crosslinking agent chosen from polyethylenic monomers comprising at least two ethylenic functions, and more particularly chosen from the elements of the group consisting of ethylene glycol dimethacrylate, tetraallyloxyethane, ethylene glycol diacrylate, diallylurea, triallylamine, trimethylolpropane triacrylate and methylenebis(acrylamide), or a mixture of these compounds.

According to a specific aspect, the gelling and/or thickening agents present in the aqueous composition (C₁) which is a subject matter of the present invention are chosen from the elements of the group consisting of:
the homopolymer of partially or completely salified acrylic acid, crosslinked with triallylamine and/or with trimethylolpropane triacrylate and/or with methylenebis(acrylamide),
the homopolymer of the sodium salt of 2-acrylamido-2-methylpropanesulfonic acid, crosslinked with triallylamine and/or with trimethylolpropane triacrylate and/or with methylenebis(acrylamide),
the copolymer of the sodium salt of 2-acrylamido-2-methylpropanesulfonic acid and of partially or completely salified acrylic acid, crosslinked with triallylamine and/or with trimethylolpropane triacrylate and/or with methylenebis(acrylamide),
the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide),
the copolymer of the sodium salt of 2-acrylamido-2-methylpropanesulfonic acid and of acrylamide, crosslinked with triallylamine and/or with trimethylolpropane triacrylate and/or with methylenebis(acrylamide),
the terpolymer of the sodium salt of 2-acrylamido-2-methylpropanesulfonic acid, of acrylamide and of partially or completely salified acrylic acid, crosslinked with triallylamine and/or with trimethylolpropane triacrylate and/or with methylenebis(acrylamide),
the terpolymer of the sodium salt of 2-acrylamido-2-methylpropanesulfonic acid, of N,N-dimethylacrylamide and of partially or completely salified acrylic acid, crosslinked with triallylamine and/or with trimethylolpropane triacrylate and/or with methylenebis(acrylamide),
a terpolymer, crosslinked by trimethylolpropane triacrylate and/or triallylamine and/or methylenebis(acrylamide), of 2-methyl-2-[(1-oxo-2-propenyl)amino]-1-propanesulfonic acid partially or completely salified in the sodium salt or ammonium salt form, in a molar proportion of greater than or equal to 60% and of less than or equal to 80%, of N,N-dimethylacrylamide, in a molar proportion of greater than or equal to 15% and of less than or equal to 39.5%, and tetraethoxylated lauryl methacrylate, in a molar proportion of greater than or equal to 0.5% and of less than or equal to 5%,
a tetrapolymer, crosslinked by trimethylolpropane triacrylate and/or triallylamine and/or methylenebis(acrylamide), of 2-methyl-2-[(1-oxo-2-propenyl)amino]-1-propanesulfonic acid partially or completely salified in the sodium salt or ammonium salt form, in a molar proportion of greater than or equal to 60% and of less than or equal to 80%, of N,N-dimethylacrylamide, in a molar proportion of greater than or equal to 15% and of less than or equal to 39%, of lauryl methacrylate, in a molar proportion of greater than or equal to and of less than or equal to 2.5%, and of stearyl methacrylate, in a molar proportion of greater than or equal to 0.5% and of less than or equal to 2.5%.

According to a more specific aspect, the gelling and/or thickening agents present in the aqueous composition (C₁) which is a subject matter of the present invention are chosen from the elements of the group consisting of xanthan gum, acacia gum exudate, the mixture of xanthan gum (Gₓ) and of gum arabic exudate (G_{A}) in a ratio by weight of xanthan gum (Gₓ) to acacia gum exudate (G_{A}) of greater than or equal to 1/3 and less than or equal to 3/1, the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide), the copolymer of the sodium salt of 2-acrylamido-2-methylpropanesulfonic acid and of acrylamide, crosslinked with triallylamine and/or with trimethylolpropane triacrylate and/or methylenebis(acrylamide), and the terpolymer, crosslinked by trimethylolpropane triacrylate and/or triallylamine and/or methylenebis(acrylamide), of 2-methyl-2-[(1-oxo-2-propenyl)amino]-1-propanesulfonic acid partially or completely salified in the sodium salt or ammonium salt form, in a molar proportion of greater than or equal to 60% and of less than or equal to 80%, of N,N dimethylacrylamide, in a molar proportion of greater than or equal to 15% and of less than or equal to 39.5%, and tetraethoxylated lauryl methacrylate, in a molar proportion of greater than or equal to 0.5% and of less than or equal to 5%.

The composition for topical use (C₁) as defined above can additionally comprise one or more auxiliary compounds chosen from solvents and cosolvents, or agents for improving skin penetration.

Another subject matter of the present invention is a composition for topical use (C′1) which is provided in the form of a gel and which comprises, per 100% of its weight:
from 50% to 80% by weight, preferably from 50% to 75% by weight and more preferentially still from 60% to 70% by weight of the lysate (Ly) as defined above,
from 0.1% to 5% by weight, preferably from 0.5% to 3% by weight, of at least one thickening and/or gelling agent, and
from 15% to 49.9% by weight, preferably from 22% to 49.5% by weight and more preferentially still from 27% to 39.5% by weight of at least one solvent.

Preferably, the solvent is chosen from the elements of the group consisting of:
the compounds of formula (Ia):

HO—[CH₂—CH(OH)—CH₂—O]ₙ—H (Ia)
in which n represents an integer greater than or equal to 1 and less than or equal to 15, more particularly greater than or equal to 1 and less than or equal to 10, more particularly greater than or equal to 1 and less than or equal to 6, more particularly greater than or equal to 1 and less than or equal to 4, more particularly equal to 1 or 2 or 3 or 4;
the compounds of formula (Ib):

Ra1-C(Rb1)(OH)—C(OH)(Rc1)(Rd1) (Ib),
in which each of the radicals Ra1, Rb1, Rc1 and Rd1 represent, independently of one another, a hydrogen atom or a saturated aliphatic radical comprising from 1 to 5 carbon atoms, or by the formula (Ib1):

Ra1-C(Rb1)(OH)—[C(Re1)(Rf1)]ₜ-C(OH)(Rc1)(Rd1) (Ib1),
in which t is equal to 1, 2 or 3 and each of the radicals Ra1, Rb1, Rc1, Rd1, Re1 and Rf1 independently represent a hydrogen atom or a saturated aliphatic radical comprising from 1 to 5 carbon atoms, it being understood that at least one of the Ra1 or Rb1 radicals and/or at least one of the Rc1 or Rd1 radicals does not represent a hydrogen atom.

More preferentially still, the solvent will be glycerol.

This composition (C′1) can be topically administrable for eliminating or reducing inflammation of the skin and/or of the scalp.

According to an even more specific aspect, the present invention relates to a topically administrable composition (C′1) for eliminating or reducing the unsightly and/or symptomatic signs linked to the inflammation of sensitive human skin and/or of the scalp, such as, for example, red patches, tightness, itching or stinging.

The compositions for topical use (C₁) and (C′₁) as defined above can additionally comprise one or more cosolvents, and agents for improving skin penetration.

Mention may be made, as examples of cosolvents optionally present in the compositions for topical use (C₁) and (C′₁) which are a subject matter of the present invention, of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols, such as ethanol, isopropanol or butanol, mixtures of water and of said organic solvents, propylene carbonate, ethyl acetate, benzyl alcohol.

Mention may be made, as examples of agents for improving skin penetration optionally present in the compositions for topical use (C₁) and (C′₁) which are a subject matter of the present invention, of glycol ethers, such as, for example, ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono(n-butyl) ether, diethylene glycol monoethyl ether (or Transcutol-P), fatty acids, such as oleic acid, fatty acid esters of glycerol, such as, for example, glyceryl behenate, glyceryl palmitate/stearate or behenoyl macroglycerides, polyoxyethylene (2) stearyl ether, polyoxyethylene (2) oleyl ether, terpenes, such as, for example, D-limonene, or essential oils, such as, for example, eucalyptus essential oil. Another subject matter of the present invention is a composition for topical use (C2) which is provided in the form of an emulsion of water-in-oil type or of an emulsion of oil-in-water type, comprising the lysate (Ly) as defined above, and preferably obtained by the implementation of a process comprising stages a) to f) as described above.

A distinction is made between oil-in-water (O/W) emulsions, the continuous phase of which consists of a hydrophilic phase, generally an aqueous phase, and the dispersed phase of which consists of a lipophilic fatty phase, and water-in-oil (W/O) emulsions, the continuous phase of which consists of a lipophilic fatty phase and the dispersed phase of which consists of a hydrophilic phase, generally an aqueous phase.

This composition (C2) can also be topically administrable for eliminating or reducing inflammation of the skin and/or of the scalp.

According to an even more specific aspect, the present invention relates to a topically administrable composition (C2) for eliminating or reducing the unsightly and/or symptomatic signs linked to the inflammation of sensitive human skin and/or of the scalp, such as, for example, red patches, tightness, itching or stinging.

It should be noted that stinging and/or tingling and/or itching and/or heating and/or red patches and/or skin discomfort and/or tightness accompany skin pathologies, such as urticaria, eczematous dermatitis, rosacea, psoriasis, herpes, photodermatoses, atopic dermatitis, contact dermatitis, lichen, prurigos, pruriginous diseases, fibroses, collagen maturation conditions, scleroderma or eczema.

Among the emulsions of water-in-oil type, a specific subject matter of the present invention is a composition for topical use (F) which is provided in the form of a water-in-oil emulsion and which comprises, per 100% of its weight:
from 60% to 90% by weight of the composition for topical use (C1) or (C′1) as defined above,
from 10% to 40% by weight of a fatty phase (A2) comprising i) at least one oil and optionally at least one wax and ii) an emulsifying system comprising at least one emulsifying surface-active agent (Si).

Preferably, the emulsifying surface-active agent (Si) will be chosen from the elements of the group consisting of alkylpolyglycoside compositions, compositions of alkylpolyglycosides and of fatty alcohols, polyglycerol esters, alkoxylated polyglycerol esters, polyglycol polyhydroxystearates, polyglycerol polyhydroxystearates and alkoxylated polyglycerol polyhydroxystearates.

In the formulation (F) for topical use which is a subject matter of the present invention, the term “oil” denotes a water-insoluble compound and/or a mixture of water-insoluble compounds which is liquid at 25° C., and more particularly:
linear alkanes comprising from 11 to 19 carbon atoms;
branched alkanes comprising from 7 to 40 carbon atoms, such as isododecane, isopentadecane, isohexadecane, isoheptadecane, isooctadecane, isononadecane or isoeicosane, or mixtures of some of them, such as those mentioned below and identified by their INCI name: C₂₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₄₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as those sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes comprising from 15 to 19 carbon atoms, said alkanes being linear alkanes, branched alkanes and cycloalkanes, and more particularly the mixture (M₁) which comprises, per 100% of its weight, a proportion by weight of branched alkanes of greater than or equal to 90% and less than or equal to 100%; a proportion by weight of linear alkanes of greater than or equal to 0% and less than or equal to 9% and more particularly less than 5%; and a proportion by weight of cycloalkanes of greater than or equal to 0% and less than or equal to 1%, for example the mixtures sold under the names Emogreen™ L15 and Emogreen™ L19;
fatty alcohol ethers of formula (II):

Z₁—O—Z₂ (II),

in which Z₁ and Z₂, which are identical or different, represent a linear or branched alkyl radical comprising from 5 to 18 carbon atoms, for example dioctyl ether, didecyl ether, didodecyl ether, dodecyl octyl ether, dihexadecyl ether, 1,3-dimethylbutyl tetradecyl ether, 1,3-dimethylbutyl hexadecyl ether, bis(1,3-dimethylbutyl) ether or dihexyl ether;
monoesters of fatty acids and of alcohols of formula (III):

R′₁—(C═O)—O—R′₂ (III),

in which R′₁—(C═O) represents a saturated or unsaturated, linear or branched, acyl radical comprising from 8 to 24 carbon atoms and R′₂ represents, independently of R′₁, a saturated or unsaturated, linear or branched, hydrocarbon chain comprising from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate or isostearyl isostearate;
diesters of fatty acids and of glycerol of formula (IV) and of formula (V):

R′₃—(C═O)—O—CH₂—CH(OH)—CH₂—O—(C═O)—R′₄ (IV)

R′₅—(C═O)—O—CH₂—CH[O—(C═O)—R′₆]—CH₂—OH (V),

in which formulae (IV) and (V) R′₃—(C═O), R′₄—(C═O), R′₅—(C═O) and R′₆—(C═O), which are identical or different, represent a saturated or unsaturated, linear or branched, acyl group comprising from 8 to 24 carbon atoms;
triesters of fatty acids and of glycerol of formula (VI):

R′₇—(C═O)—O—CH₂—CH[O—(C═O)—R″₈]—CH₂—O—(C═O)—R″₉ (VI),

in which R′₇—(C═O), R′₈—(C═O) and R′₉—(C═O), which are identical or different, represent a linear or branched, saturated or unsaturated, acyl group comprising from 8 to 24 carbon atoms;
vegetable oils, such as phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy oil, red kuri squash oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil or oils resulting from flowers or vegetables;
ethoxylated vegetable oils.

Preferably, the composition according to the invention comprises at least one oil chosen from the elements of the group consisting of castor oil, liquid paraffins, cocoyl caprylate/caprate, isopropyl myristate and capric/caprylic triglyceride.

The fatty phase (A2) optionally comprises wax. In the formulation (F) for topical use which is a subject matter of the present invention, the term “wax” denotes a water-insoluble compound and/or a mixture of water-insoluble compounds which is solid at 35° C.

Such a wax is more particularly chosen from beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; vegetable waxes; fatty alcohols and fatty acids which are solid at ambient temperature; glycerides which are solid at ambient temperature.

In the definition of the formulation (F) which is a subject matter of the present invention, the term “alkylpolyglycoside compositions” denotes a composition (A) represented by the formula (VII):

R₁—O-(G)ₓ—H (VII)

in which x represents a decimal number of between 1.05 and 5, G represents the residue of a reducing sugar and R₁ represents a saturated or unsaturated, linear or branched, aliphatic hydrocarbon radical, optionally substituted with one or more hydroxyl groups, comprising from 12 to 36 carbon atoms, said composition (A) consisting of a mixture of compounds represented by the formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x.

The term “saturated or unsaturated, linear or branched, aliphatic hydrocarbon radical, comprising from 12 to 36 carbon atoms, optionally substituted with one or more hydroxyl groups” denotes, for the radical R₁ in the formula (VII) as defined above:
saturated linear alkyl radicals, for example the n-dodecyl, n-tridecyl, n-tetradecyl, n-pentadecyl, n-hexadecyl, n-heptadecyl, n-octadecyl, n-nonadecyl, n-eicosyl or n-docosyl radicals;
unsaturated linear radicals, such as the dodecenyl, tridecenyl, tetradecenyl, pentadecenyl, hexadecenyl, heptadecenyl, octadecenyl, nonadecenyl, eicosenyl, docosenyl, 4-dodecenyl or 5-dodecenyl radicals;
saturated or unsaturated, linear or branched, aliphatic radicals comprising from 12 to 36 carbon atoms substituted by one or two hydroxyl groups, such as the hydroxydodecyl, hydroxytetradecyl, hydroxyhexadecyl, hydroxyoctadecyl, hydroxyeicosyl or hydroxydocosyl radicals, for example the 12-hydroxyoctadecyl radical;
radicals resulting from the isoalkanols of formula (1):

(CH₃)(CH₃)CH—(CH₂)ᵣ—CH₂—OH (1)

in which r represents an integer between 8 and 20, for example the isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals;
branched alkyl radicals, resulting from Guerbet alcohols, of formula (2):

CH(C₅H₂ₛ₊₁)(CₜH₂ₜ₋₄)—CH₂—OH (2)

in which t is an integer between 6 and 18, s is an integer between 4 and 18 and the sum s+t is greater than or equal to 10 and less than or equal to 22, for example the 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals.

According to a specific aspect, in the definition of the formula (VII) as defined above, R₁ represents a saturated or unsaturated, linear or branched, aliphatic hydrocarbon radical comprising from 12 to 24 carbon atoms.

The term “reducing sugar” in the definition of the formula (VII) as defined above denotes saccharide derivatives which do not exhibit, in their structures, a glycoside bond established between an anomeric carbon and the oxygen of an acetal group, as are defined in the reference work: “Biochemistry”, Daniel Voet/Judith G. Voet, p. 250, John Wiley & Sons, 1990. The oligomeric structure (G)ₓ can exist in any isomeric form, whether it concerns optical isomerism, geometrical isomerism or positional isomerism; it can also represent a mixture of isomers.

In the formula (VII) as defined above, the R₁—O— group is bonded to G via the anomeric carbon of the saccharide residue, so as to form an acetal function.

According to a specific aspect in the definition of the formula (VII) as defined above, G represents the residue of a reducing sugar chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran or tallose; and more particularly G represents the residue of a reducing sugar chosen from the residues of glucose, xylose and arabinose.

According to an even more specific aspect, in the definition of the formula (VII), x represents a decimal number greater than or equal to 1.05 and less than or equal to 2.5, more particularly greater than or equal to 1.05 and less than or equal to 2.0 and more particularly still greater than or equal to 1.25 and less than or equal to 2.0.

According to an even more specific aspect, in the definition of the formula (VII) as defined above, R₁ represents the radical chosen from at least one of the elements of the group consisting of the n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals; G represents the residue of a reducing sugar chosen from the residues of glucose and xylose and x represents a decimal number greater than or equal to 1.05 and less than or equal to 2.5. According to an even more specific aspect, the formulation (F) as defined above is characterized in that said emulsifying system consists of an alkylpolyglycoside composition (A) represented by the formula (VII):

R₁—O-(G)ₓ-H (VII)

in which x represents a decimal number between 1.05 and 2.5, G represents the residue of xylose and R₁ represents the 2-octyldodecyl radical, said composition (A) consisting of a mixture of compounds represented by the formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x.

In the definition of the composition (F) which is a subject matter of the present invention, the term “composition of alkylpolyglycosides and of fatty alcohols” denotes a composition (B) comprising, per 100% of its weight:
from 10% to 50% by weight, more particularly from 15% to 40% by weight and more particularly still from 20% to 30% by weight of at least one composition (A) represented by the formula (VII) as defined above,
from 90% to 50% by weight, more particularly from 85% to 60% by weight and more particularly still from 80% to 70% by weight of at least one fatty alcohol of formula (VIII):

R′₁—OH (VIII),

in which R′₁, which is identical to or different from R₁, represents a saturated or unsaturated, linear or branched, aliphatic hydrocarbon radical, optionally substituted with one or more hydroxyl groups, comprising from 12 to 36 carbon atoms.

According to a specific aspect, in the definition of the formula (VII) representing the composition (A) included in the composition (B), R₁ represents the radical chosen from the n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl or 2-decyltetradecyl radicals, G represents the residue of a reducing sugar chosen from the residues of glucose and xylose and x represents a decimal number greater than or equal to 1.05 and less than or equal to 2.5.

According to a more specific aspect, in the definition of the formula (VII) representing the composition (A) included in the composition (B), R₁ represents the 2-octyldodecyl radical, G represents the residue of xylose and x represents a decimal number greater than or equal to 1.05 and less than or equal to 2.5.

According to a more specific aspect, in the definition of the fatty alcohol of formula (VIII) as defined above, R′₁ represents a radical chosen from the n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl or 2-decyltetradecyl radicals; R′₁ very particularly represents the 2-octyldodecyl radical. According to an even more specific aspect, a subject matter of the invention is a formulation (F) as defined above, characterized in that said emulsifying system consists of a composition (B) comprising, per 100% of its weight:
from 10% to 50% by weight of at least one alkylpolyglycoside composition (A) represented by formula (VII):

R₁—O-(G)ₓ-H (VII)

in which x represents a decimal number between 1.05 and 2.5, G represents the residue of xylose and R₁ represents the 2-octyldodecyl radical, said composition consisting of a mixture of compounds represented by the formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x; and
   from 90% to 50% by weight of at least one fatty alcohol of formula (VIII):

R′₁—OH (VIII),

in which represents the 2-octyldodecyl radical.

In the definition of the formulation (F) which is a subject matter of the present invention, the term “polyglycerol ester” denotes a compound of formula (IX):

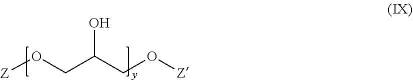

in which Z represents an acyl radical of formula R₂—C(═O)—, in which R₂ represents a saturated or unsaturated, linear or branched, aliphatic hydrocarbon radical, comprising from 11 to 35 carbon atoms, and more particularly a radical chosen from the dodecanoyl, tetradecanoyl, hexadecanoyl, octadecanoyl, eicosanoyl, docosanoyl, oleyl, linoleyl, linolenoyl or isostearyl radicals, Z′ represents the acyl radical of formula R₂—C(═O)— as defined above, with Z′ identical to or different from Z, or the hydrogen atom, and y represents an integer greater than or equal to 2 and less than or equal to 20.

According to a more specific aspect, the compound of formula (IX) is chosen from the elements of the group consisting of decaglyceryl oleate, decaglyceryl isostearate, decaglyceryl monolaurate, decaglyceryl monolinoleate and decaglyceryl monomyristate.

In the definition of the formulation (F) which is a subject matter of the present invention, the term “alkoxylated polyglycerol esters” denotes a compound of formula (X):

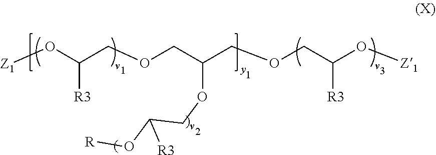

in which Z₁ represents an acyl radical of formula R′₂—C(═O)—, in which R′₂ represents a saturated or unsaturated, linear or branched, aliphatic hydrocarbon radical, comprising from 11 to 35 carbon atoms, and more particularly a radical chosen from the dodecanoyl, tetradecanoyl, hexadecanoyl, octadecanoyl, eicosanoyl, docosanoyl, oleyl, linoleyl, linolenoyl or isostearyl radicals, represents the acyl radical of formula R′₂—C(═O)— as defined above, with identical to or different from Z₁, or the hydrogen atom, R₃ represents the hydrogen atom, the methyl radical or the ethyl radical, y₁ represents an integer greater than or equal to 2 and less than or equal to 20, v₁, v₂ and v₃, which are identical or different, represent an integer greater than or equal to 0 and less than or equal to 50, and the sum [(y₁. v₁)+(y₁. v₂)+v₃] is an integer greater than or equal to 1 and less than or equal to 50.

In the definition of the composition (F) which is a subject matter of the present invention, the term “polyglycol polyhydroxystearates” denotes a compound of formula (XI):

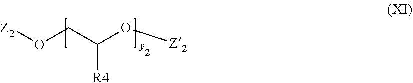

in which y₂ represents an integer greater than or equal to 2 and less than or equal to 50, R₄ represents the hydrogen atom, the methyl radical or the ethyl radical, Z₂ represents a radical of formula (XII):

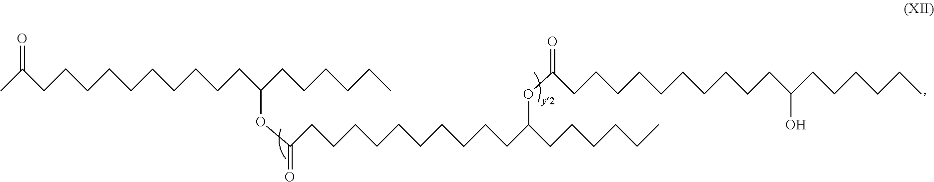

in which y′₂ represents an integer greater than or equal to 0 and less than or equal to 10, more particularly greater than or equal to 1 and less than or equal to 10, and Z′₂ represents a radical of formula (XII) as defined above, with Z′₂ identical to or different from Z₂, or the hydrogen atom.

In the definition of the formulation (F) which is a subject matter of the present invention, the term “polyglycerol polyhydroxystearate” denotes a compound represented by the formula (XIII):

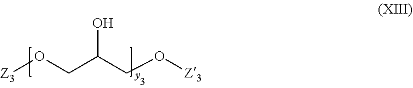

in which Z₃ represents a radical of formula (XII) as defined above, Z′₃ represents a radical of formula (XII) as defined above, with Z′₃ identical to or different from Z₃, or the hydrogen atom, and y₃ represents an integer greater than or equal to 2 and less than or equal to 20.

In the definition of the formulation (F) which is a subject matter of the present invention, the term “alkoxylated polyglycerol polyhydroxystearate” denotes a compound represented by the formula (XIV):

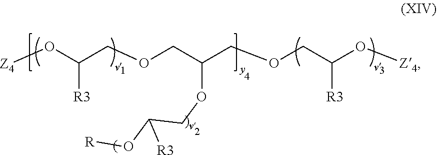

in which Z₄ represents a radical of formula (XII) as defined above, Z′₄ represents a radical of formula (XII) as defined above, with Z′₄ identical to or different from Z₄, or the hydrogen atom, y₄ represents an integer greater than or equal to 2 and less than or equal to 20, v′₁, v′₂ and v′₃, which are identical or different, represent an integer greater than or equal to 0 and less than or equal to 50, and the sum [(y₄. v′₁)+(y₄. v′₂)+v′₃] is an integer greater than or equal to 1 and less than or equal to 50.

According to another specific aspect, a subject matter of the invention is a formulation (F) as defined above, characterized in that said emulsifying system (S) consists of a composition (D) comprising, per 100% of its weight:

from 15% to 25% by weight of at least one composition (A) represented by the formula (VII):

R₁—O-(G)ₓ-H (VII)

in which x represents a decimal number between 1.05 and 2.5, G represents the residue of xylose and R₁ represents the 2-octyldodecyl radical, said composition (A) consisting of a mixture of compounds represented by the formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x;
from 55% to 65% by weight of at least one fatty alcohol of formula (VIII):

R′₁—OH (VIII),

in which R′₁ represents the 2-octyldodecyl radical;
from 10% to 30% by weight of at least one polyglycol polyhydroxystearate represented by the formula (XI):

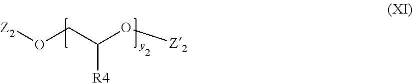

in which y₂ represents an integer greater than or equal to 2 and less than or equal to 50, R₄ represents the hydrogen atom, the methyl radical or the ethyl radical, Z₂ represents a radical of formula (XII):

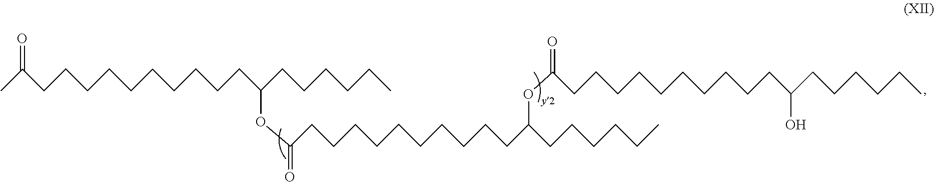

in which y′₂ represents an integer greater than or equal to 0 and less than or equal to 10, more particularly greater than or equal to 1 and less than or equal to 10, and Z′₂ represents a radical of formula (XII) as defined above, with Z′₂ identical to or different from Z₂, or the hydrogen atom.

According to another specific aspect, a subject matter of the invention is a formulation (F) as defined above, characterized in that the dynamic viscosity of said formulation (F), measured at a temperature of 25° C. using a Brookfield viscometer of LVT type at a speed of 6 revolutions/minute, is greater than or equal to 500 mPa·s and less than or equal to 40 000 mPa·s.

Another subject matter of the present invention is a composition for topical use (F′) which is provided in the form of an oil-in-water emulsion and which comprises, per 100% of its weight:
from 50% to 90% by weight, preferably from 60 to 90% by weight and more preferentially still from 70 to 90% by weight of a cosmetically acceptable aqueous phase (A1), said aqueous phase (A1) comprising, per 100% of its own weight, from to 10% by weight of the lysate (Ly) as defined above,
from 10% to 50% by weight, preferably from 10% to 40% by weight and more preferentially still from 10% to 30% by weight of a fatty phase (G1) comprising, per 100% of its own weight:
   from 0.5% to 20% by weight, preferably from 1% to 15% by weight, of at least one surface-active agent of oil-in-water type (S′1),
   from 80% to 99.5% by weight of at least one oil and/or one wax.

Within the meaning of the present invention, the term “oil” present in the fatty phase (G₁) of the composition (F′) existing in the form of an emulsion of oil-in-water type as defined above denotes chemical substances or mixtures of chemical substances which are insoluble in water and which exist under a liquid appearance at a temperature of 25° C.

Within the meaning of the present invention, the term “wax” present in the fatty phase (G₁) of the composition (F′) existing in the form of an emulsion of oil-in-water type as defined above denotes chemical substances or mixtures of chemical substances which are insoluble in water and which exist under a solid appearance at a temperature of 45° C.

Within the meaning of the present invention, the term “surface-active agent of oil-in-water type (S′1)” present in the fatty phase (G₁) of the composition (F′) existing in the form of an emulsion of oil-in-water type as defined above denotes the chemical substance or the mixture of chemical substances which makes it possible to stabilize the droplets of said fatty phase (G₁) in dispersion in the continuous aqueous phase (A₁).

Mention may be made, as surface-active agent of oil-in-water type (S′1) present in the fatty phase (G1) of the emulsion (F′) of oil-in-water type as defined above, for example, of:
polysorbates resulting from the ethoxylation reaction between one molar equivalent of sorbitan esters and between 5 and 20 molar equivalents of ethylene oxide, and more particularly between one molar equivalent of sorbitan laurate or of sorbitan palmitate or of sorbitan stearate or of sorbitan isostearate or of sorbitan oleate and between 5 and 20 molar equivalents of ethylene oxide, more particularly 5 or 10 or 12 or 15 or 20 molar equivalents of ethylene oxide;
the products resulting from the ethoxylation reaction between one molar equivalent of a fatty acid, such as, for example, palmitic acid, myristic acid, lauric acid, stearic acid, isostearic acid or oleic acid, and between 5 and 40 molar equivalents of ethylene oxide;
the products resulting from the esterification reaction between a fatty acid, such as, for example, palmitic acid, myristic acid, lauric acid, stearic acid, isostearic acid, oleic acid, arachidic acid or behenic acid, and between 4 and 20 molar equivalents, more particularly between 3 and 10 molar equivalents, of glycerol.

Within the meaning of the present invention, the term “fatty phase (G1)” denotes a fatty substance or a mixture of fatty substances which is insoluble in water and/or in mixtures of water and of polar solvents. Such a “fatty phase” can comprise oils and/or waxes as defined above.

Mention may be made, among the constituent elements of the fatty phase (G1) present in the formulation (F′), of the constituent oils of the fatty phase (A2) such as described above for the preparation of the formulation (F) and of silicone oils, such as dimethylpolysiloxanes, methylphenylpolysiloxanes, silicones modified by amines, silicones modified by fatty acids, silicones modified by alcohols, silicones modified by alcohols and fatty acids, silicones modified by polyether groups, modified epoxy silicones, silicones modified by fluorinated groups, cyclic silicones and silicones modified by alkyl groups.

Mention may be made, among the constituent elements of the fatty phase (G1) present in the formulation (F′), of the constituent waxes of the fatty phase (A2) such as described above for the preparation of the formulation (F).

According to a specific aspect, in the formulation (F′) which is a subject matter of the present invention, the fatty phase (G1) additionally comprises, per 100% of its own weight, from 5% to 30% by weight, more particularly from 5% to 25% by weight and more particularly still from 10% to 25% by weight of at least one agent for protecting against the ultraviolet radiation of the sun.

The term “agent for protecting against the ultraviolet radiation of the sun” denotes in particular, in the definition of the composition (F′) for topical use which is provided in the form of an emulsion of oil-in-water type and which is a subject matter of the present invention, pigments, organic sunscreens and inorganic sunscreens.

Examples of pigments used as agent for protecting against the ultraviolet radiation of the sun are titanium dioxide, brown iron oxides, yellow iron oxides, black iron oxides or red iron oxides or also white or colored pearlescent pigments, such as titanium oxide-coated micas.

Examples of organic sunscreens used as agent for protecting against the ultraviolet radiation of the sun are:
those of the family of the derivatives of benzoic acid, such as para-aminobenzoic acids (PABAs), in particular monoglycerol esters of PABA, ethyl esters of N,N-propoxy-PABA, ethyl esters of N,N-diethoxy-PABA, ethyl esters of N,N-dimethyl-PABA, methyl esters of N,N-dimethyl-PABA, butyl esters of N,N-dimethyl-PABA;
those of the family of the derivatives of anthranilic acid, such as homomenthyl N-acetylanthranilate;
those of the family of the derivatives of salicylic acid, such as amyl salicylate, homomenthyl salicylate, ethylhexyl salicylate, phenyl salicylate, benzyl salicylate, p-isopropanolphenyl salicylate;
those of the family of the derivatives of cinnamic acid, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnannate, 2-ethylhexyl α-cyano-β-phenylcinnannate, glyceryl di-para-methoxy mono-2-ethylhexanoyl cinnamate;
those of the family of the derivatives of benzophenone, such as 2,4-dihydroxybenzophenone, 2,2′-dihydroxy-4-methoxybenzophenone, 2,2′,4,4′-tetra hydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2-carboxylate, 2-hydroxy-4-(n-octyloxy)benzophenone, 4-hydroxy-3-carboxybenzophenone; 3-(4′-methylbenzylidene)-d,l-camphor, 3-benzylidene-d,l-camphor, camphor benzalkonium methosulfate; urocanic acid, ethyl urocanate;
those of the family of the derivatives of sulfonic acid, such as 2-phenylbenzimidazole-5-sulfonic acid and its salts; the family of the derivatives of triazine, such as hydroxyphenyl triazine, ethylhexyloxyhydroxyphenyl-4-methoxyphenyltriazine, 2,4,6-trianilino(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methylphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis[4-(2-ethylhexyloxy)-2-hydroxyphenyl]-6-(4-methoxyphenyl)-1,3,5-triazine, 2,4,6-tris[4-(2-ethylhexyloxycarbonyl)anilino]-1,3,5-triazine, 2-ethyl hexyl dimethoxybenzylidene dioxoimidazolidine propionate, the family of the derivatives of diphenylacrylate, such as 2-ethylhexyl 2-cyano-3,3-diphenyl-2-propenoate or ethyl 2-cyano-3,3-diphenyl-2-propenoate;
those of the family of the polysiloxanes, such as benzylidene siloxane malonate.

Examples of inorganic sunscreens used as agent for protecting against the ultraviolet radiation of the sun are: titanium oxides, zinc oxides, cerium oxide, zirconium oxide, yellow, red or black iron oxides, chromium oxides. These inorganic filters may or may not be micronized, may or may not have been subjected to surface treatments and may optionally be presented in the form of aqueous or oily predispersions.

The agent for protecting against the ultraviolet radiation of the sun will preferably be chosen from the elements of the group consisting of titanium dioxide, 2,4-dihydroxybenzophenone, 2-(4-diethylamino-2-hydroxybenzoyl)benzoic acid hexyl ester, 2,4-bis[4-(2-ethylhexyloxy)-2-hydroxyphenyl]-6-(4-methoxyphenyl)-1,3,5-triazine, 2,4,6-tris[4-(2-ethylhexyloxycarbonyl)anilino]-1,3,5-triazine and 2-ethylhexyl dimethoxybenzylidene dioxoimidazolidine propionate.

The expression “for topical use” used in the definition of the composition (E1) provided in the form of an emulsion of oil-in-water type as defined above means that said composition is formulated to make possible its application to the skin, the hair, the scalp or the mucus membranes, whether it is a direct application in the case of a cosmetic, dermocosmetic, dermopharmaceutical or pharmaceutical composition or an indirect application, for example in the case of a body hygiene product in the form of a textile or paper wipe, or sanitary products intended to be in contact with the skin or the mucus membranes.

The expression “cosmetically acceptable” used in the definition of the aqueous phase (A₁) of the composition (E1) provided in the form of an emulsion of oil-in-water type means, according to the Council of the European Economic Community Directive No. 76/768/EEC of Jul. 27, 1976, amended by Directive No. 93/35/EEC of Jun. 14, 1993, any substance or preparation intended to be brought into contact with the various parts of the human body (epidermis, body hair and head hair system, nails, lips and genitals) or with the teeth and mucus membranes of the mouth, for the purpose, exclusively and mainly, of cleansing them, scenting them, modifying the appearance thereof and/or correcting body odors thereof and/or protecting them or keeping them in good condition.

The compositions (F) and (F′) can be topically administrable for reducing or eliminating inflammation of the skin and/or of the scalp.

According to an even more specific aspect, the present invention relates to a topically administrable composition (F) and a topically administrable composition (F′) for eliminating or reducing the unsightly and/or symptomatic signs linked to the inflammation of sensitive human skin and/or of the scalp, such as, for example, red patches, tightness, itching or stinging.

It should be noted that stinging and/or tingling and/or itching and/or heating and/or red patches and/or skin discomfort and/or tightness accompany skin pathologies, such as urticaria, eczematous dermatitis, rosacea, psoriasis, herpes, photodermatoses, atopic dermatitis, contact dermatitis, lichen, prurigos, pruriginous diseases, fibroses, collagen maturation conditions, scleroderma or eczema.

### DESCRIPTION OF THE PREFERRED EMBODIMENTS

1) Preparation of a Lysate According to the Invention (Ly)

A lysate (Ly) of dedifferentiated cells of the plant Helichrysum stoechas is prepared by carrying out the process described above and more particularly by carrying out:
a stage a) of preparation of the sterile sample of Helichrysum stoechas, as described above, using Helichrysum stoechas leaves cut into fragments of a few millimeters, then sterilized by successive passage:
through a bath of 70% ethanol and Tween™ 80 (at 0.05%) for five minutes
through a bath of 1% bleach for five minutes.

The leaves are subsequently rinsed in sterile distilled water three times in succession.
A stage b) of callogenesis, as described above, with a callogenesis medium, the constitution of which is described in table 1 above, with an incubation phase with a duration of three weeks at a temperature of 20 to 25° C. in the light.
A stage c) of suspending the dedifferentiated cells, as described above, with a culture medium, the composition of which is described in table 2 above, for a period of time of 14 to 30 days, at a temperature of 20 to 25° C., in daylight, stirred by a stirrer of orbital type at a speed of 100 revolutions/minute.
A stage d) of selection of a fine suspension as described above, with a withdrawal of ⅙ to ¼ of culture obtained in stage c), to which is added a volume of suspension medium as described in table 2 and in a volume adjusted to the volume of inoculum available in the container.
The new suspension is cultured in a climatically controlled chamber for a period of time of 14 days, at a temperature of 20 to 25° C., in daylight, stirred by stirring of orbital type at a speed of 100 revolutions/minute.
A stage e) of production of biomass as described above, with a withdrawal of ⅙ to ¼ of the medium obtained in stage d), to which is added a volume of suspension medium as described in table 2 and in a volume adjusted to the volume of inoculum available in the container.
The new suspension is cultured in a climatically controlled chamber for a period of time of 14 days in a wave bioreactor with a capacity of between 5 liters and 1000 liters, at a temperature of 20 to 25° C., in daylight, stirred by stirring of orbital type at a speed of 25 revolutions/minute.
A stage f) of high-pressure homogenization of the medium obtained on conclusion of stage e), by passage through a high-pressure homogenizer of the GEA brand, at a pressure of 500 bars on one pass at a temperature below 40° C.

A lysate (Ly) is thus obtained comprising, per 100% of its weight:
90-98% by weight of water
10-2% by weight of dry matter

A stage g) of stabilization of the medium obtained in stage f), by mixing 60% by weight of the medium obtained in stage f) with 0.8% by weight of a thickening agent sold under the brand name Solagum™ AX, 37.9% by weight of glycerol, 0.3% of potassium sorbate and 1% of sodium benzoate, brought to a value of the pH of 5.3.

A composition C_{A} is thus obtained comprising, per 100% of its weight:
60% by weight of lysate (Ly)
37.9% by weight of glycerol
0.8% by weight of Solagum™ AX
0.3% by weight of potassium sorbate
1% by weight of sodium benzoate.

2) Experimental use of the biological effects of a lysate of dedifferentiated cells of the plant Helichrysum stoechas according to the invention

As already mentioned, the lysate as defined above and obtained by the implementation of a process comprising stages a) to f) as are described above makes it possible to resolve the inflammation of human skin, more particularly dry, reactive and/or sensitive human skin, and thus to maintain a good level of hydration contributing to reducing feelings of discomfort/tightness, thus making it possible to soothe, to contribute to the comfort of human skin, more particularly dry, reactive and/or sensitive human skin.

The specific technical effect of the lysate as defined above and obtained by the implementation of a process comprising stages a) to f) as are described above has been demonstrated over several in vitro and in vivo study models.

A—Soothing Effect

A.1) The soothing effect of the lysate (Ly) as defined above was demonstrated using a model of coculturing of primary human keratinocytes with dendritic cells.

Dendritic cells form part of the cells of the cutaneous immune system, and they are involved in the early inflammatory response. This model is targeted at mimicking the interactions which take place naturally in the skin between the keratinocytes and the cells of the immune system, and it makes it possible to evaluate the impact of active principles on the processes of resolution of inflammation.

Briefly, normal human keratinocytes (NHEK) are inoculated in inserts and cultured without contact with dendritic cells (DC) inoculated in the bottom of the wells. The cells are pretreated with the composition C_{A} or glycerol. After 20 hours of incubation, the inflammatory stimulus (mixture of phorbol 12-myristate 13-acetate (PMA) and of calcium ionophore A23187), polyunsaturated fatty acids (PUFAs, lipid precursors of inflammation mediators, eicosanoids) and the compounds (composition C_{A} or glycerol) were again added and incubated for 1 hour. Subsequently, the culture supernatants were collected for analysis of lipid mediators.

The experiments were replicated three times.

For each culture supernatant, the following were analyzed:
the amount of 5-HETE (5-HydroxyEicosaTetraEnoic acid), proinflammatory mediator
the amount of LTB4 (leukotriene B4), proinflammatory mediator
the amount of 12-HETE (12-HydroxyEicosaTetraEnoic acid), proresolving mediator
the amount of 15-HETE (15-HydroxyEicosaTetraEnoic acid), proresolving mediator
the amount of 17-HDOHE ((+/−)-17-Hydroxy-4Z,7Z,10Z,13Z,15E,19Z-docosahexaenoic acid), proresolving mediator.

The amounts of 5-HETE, LTB4, 12-HETE, 15-HETE and 17-HDOHE are measured by the use of an LC-MS/MS method using a UPLC system (Agilent LC1290 Infinity) coupled to an Agilent 6490 triple quadrupole mass spectrometer (Agilent Technologies) equipped with electrospray ionization operating in negative mode, and are expressed in picograms per culture well (pg/well).

The values obtained were expressed as mean+/−sem [standard error of the mean=standard deviation/square root (number of values)].

For each condition, the percentages of activation were calculated as follows:

% activation=[mean (condition)]/[mean (stimulated cells)]×100−100.
stimulated cells: PMA/A23187/PUFA-treated cells in the case where the condition studied is the treatment with glycerol.
stimulated cells: cells treated with PMA/A23187/PUFA+0.1% DMSO in the case where the condition studied is the treatment with the composition C_{A}.

The statistical analysis was carried out by a Student's test and a significance threshold was set at 5%, the conditions being compared in pairs. A difference between the effectiveness of two products was considered:
significant if p<0.05;
“at the limit of significance” if 0.05≤p<0.1;
and not significant if p>0.1.

The results are presented in table 4 below:

TABLE 3

| | 5-HETE | | | LTB4 | | |
|---|---|---|---|---|---|---|
| | (pg/well) | | | (pg/well) | | |
| | Mean +/− | % | | Mean +/− | % | |
| | SEM | activation | | SEM | activation | |
| Untreated cells | 3.1 | — | | 0 | — | |
| (control) | | | | | | |
| PMA/A23187/PUFA | 472 +/− 26 | — | | 213 +/− 8 | — | |
| Glycerol (0.05%) | 1152 +/− 201 | 144%* | | 400 +/− 38 | 88%* | |
| PMA/A23187/PUFA + | 285 +/− 14 | — | | 108 +/− 12 | — | |
| 0.1% DMSO | | | | | | |
| Composition C_{A} (0.1%) | 203 +/− 15 | −29%* | | 83 +/− 4 | −24%* | |
| | 12-HETE | | 15-HETE | | 17-HDOHE | |
| | (pg/well) | | (pg/well) | | (pg/well) | |
| | Mean +/− | % | Mean +/− | % | Mean +/− | % |
| | SEM | activation | SEM | activation | SEM | activation |
| Untreated cells | 0 | — | 40.5 | — | 17.3 | — |
| (control) | | | | | | |
| PMA/A23187/PUFA | 9 +/− 2 | — | 168 +/− 14 | — | 14768 +/− 464 | — |
| Glycerol (0.01%) | 15 +/− 1 | 58%¹⁵ | 273 +/− 26 | 63%* | 16654 +/− 1409 | +13% |
| PMA/A23187/PUFA + | 7 +/− 2 | — | 103 +/− 15 | — | 10183 +/− 478 | — |
| 0.1% DMSO | | | | | | |
| Composition C_{A} (0.01%) | 10 +/− 0 | 41%* | 152 +/− 13 | 48% | 12837 +/− 125 | 26%** |

Determination of the Activation of the Secretion of Pro- and Antiinflammatory Mediators Following Stimulation with PMA/A23187/PUFA. LS 0.1<p<0.05, *p<0.05, **p<0.01

In conclusion, the composition C_{A} is capable, on the one hand, of reducing the production of proinflammatory molecules and, on the other hand, of stimulating the secretion of several proresolving mediators and thus of promoting the resolution of inflammation (production of lipoxins, type-D resolvins and protectins), thus making it possible to reduce inflammation and to promote the return to homeostasis.

A.2) The soothing effect on impaired skin was also shown using a model of explants with an impaired skin barrier. This detrimental change in the barrier function is induced by a piece of adhesive tape applied to the skin and then removed (tape stripping).

This method consists in applying a piece of adhesive tape (“D-Squame” brand) to the surface of the skin and in pressing it on the surface of the skin, and then in removing it. The first applications remove a complete cell layer of corneocytes. The amount of corneocytes decreases on the adhesive tape as the applications progress.

The “D-Squame” adhesive tapes are applied to the explants, care being taken that they are always carried out at the same place and in the same way, with a constant pressure of the thumb. The adhesive tape is subsequently removed in a smooth, rapid and unidirectional movement in order to obtain a homogeneous removal of the corneocytes. This model makes it possible to evaluate the impact of cosmetic formulations on the physiology of the epidermides, the barrier function of which is detrimentally affected.

The experiments were replicated twice.

The values were expressed as mean+/−sd [standard deviation].

For each condition, the percentages of stimulation or of protection were calculated as follows:

% Restoration=100×[mean (composition C_{A})−mean (impaired epidermides)]/[mean (healthy epidermides)−mean (impaired epidermides)].

The method used is described in detail below.

The explants exhibiting an impaired barrier function (by the tape stripping method) and characterized by an insensible water loss of greater than 35 g/m²·h are treated one hour after the detrimental change in the barrier function, and every day for five days, with a formulation (F₁) comprising 1% of the composition C_{A} and the constitution by weight of which is as follows.

| Ingredients | Content as percentage |
|---|---|
| | by weight |
| water | q.s. for 100% |
| Glycerol | 1% |
| Montanov™ L⁽¹⁾ | 1 |
| Simulsol™ 165⁽²⁾ | 1 |
| C8-C10 Triglycerides | 3 |
| Caprylic/Capric Triglycerides | |
| DUB™ DNPG⁽³⁾ | 7 |
| Neopentyl glycol diheptanoate | |
| Sepimax™ Zen⁽⁴⁾ | 1 |
| Polyacrylate Crosspolymer-6 | |
| Sepinov™ EMT 10⁽⁵⁾ | 0.5 |
| Hydroxyethyl Acrylate/Sodium | |
| Acryloyldimethyl Taurate Copolymer | |
| Euxy\|™ PE 9010⁽⁶⁾ | 1 |
| Phenoxylethanol & Ethylhexylglycerin | |
| Sensiva™ PA40⁽⁷⁾ 3-Phenylpropan-1-ol and | 0.5 |
| Octane-1,2-diol and Propane-1,3,-diol | |
| Composition C_{A} | 1 |

⁽¹⁾Montanov™ L (INCI name: C14-22 Alcohols & C12-20 Alkyl Glucoside) is an emulsifying composition, intended to stabilize emulsions, and used in the preparation of cosmetic formulations.
⁽²⁾Simulsol™ 165 (INCI name: PEG-100 Stearate and Glyceryl Stearate) is an emulsifying composition, intended to stabilize emulsions, in particular oil-in-water emulsions, and used in the preparation of cosmetic formulations.
⁽³⁾DUB™ DNPG (INCI name: Neopentyl Glycol Diheptanoate) is an ester used as a fatty phase and/or emollient agent in the preparation of cosmetic formulations.
⁽⁴⁾Sepimax™ Zen (INCI name: Polyacrylate Crosspolymer-6) is a crosslinked anionic polyelectrolyte used as thickening and/or emulsifying and/or stabilizing agent in the preparation of cosmetic formulations.
⁽⁵⁾Sepinov™ EMT 10 (INCI name: Hydroxyethyl Acrylate/Sodium Acryloyldimethyl Taurate Copolymer) is a crosslinked anionic polyelectrolyte used as thickening and/or emulsifying and/or stabilizing agent in the preparation of cosmetic formulations.
⁽⁶⁾Euxy|™ PE 9010 (INCI name: Phenoxyethanol & Ethylhexylglycerin) is a preservative used in the preparation of cosmetic formulations.
⁽⁷⁾Sensiva™ PA40 (INCI name: 3-Phenylpropan-1-ol and Octane-1,2-diol and Propane-1,3-diol) is a preservative used in the preparation of cosmetic formulations.

The tissues are digested in an aqueous and organic mixture. Protein precipitation is performed. Several successive extractions are subsequently carried out in order to purify and concentrate the sample.

The extracts are subsequently analyzed using an LC/MS-MS chromatographic method making possible the quantitative analysis of eicosanoids, lipid mediators of inflammation.

The LC/MS-MS analysis was carried out on an UltiMate 3000 HPLC system (ThermoScientific) coupled to an MSQ Plus mass spectrometer (ThermoScientific), equipped with electrospray ionization operating in negative mode.

For each extract, the following are analyzed:
the amount of 8-HETE (8-HydroxyEicosaTetraEnoic acid), proinflammatory mediator
the amount of PGE2 (Prostaglandin E2), proinflammatory mediator
the amount of 15-HETE (15-HydroxyEicosaTetraEnoic acid), proresolving mediator

The amounts of 8-HETE, PGE2 and 15-HETE are expressed in arbitrary units per milligram of proteins (AU/mg of proteins).

The results are presented in table 4 below:

TABLE 4

| | 8-HETE (AU/mg | | PGE2 (AU/mg of | |
|---|---|---|---|---|
| | of proteins) | | proteins) Mean | |
| | Mean +/− SD | % Restoration | +/− SD | % Restoration |
| Healthy | 11.5 +/− 1.9 | — | 8.7 +/− 1.3 | — |
| explants | | | | |
| Impaired | 17.3 +/− 2.5 | — | 17.2 +/− 2.0 | — |
| explants | | | | |
| Impaired | 14.1 +/− 0.6 | 56% | 13.4 +/− 1.1 | 45% |
| explants + | | | | |
| Formulation (F1) | | | | |
| having 1% of | | | | |
| the Composition | | | | |
| CA | | | | |

Restoration of the secretion of pro-inflammatory mediators following the detrimental change
in the barrier function by tape stripping (no statistics - n = 2)

| | 15-HETE (AU/mg of proteins) | |
|---|---|---|
| | Mean +/− SD | % Restoration |
| Healthy explants | 12.3 +/− 1.5 | — |
| Impaired explants | 5.9 +/− 1.0 | — |
| Impaired explants + | 8.3 +/− 1.1 | 38% |
| Formulation (F1) having 1% | | |
| of the Composition CA | | |

In conclusion, the composition C A is capable, on the one hand, of reducing the production of proinflammatory mediator PGE2 and, on the other hand, of stimulating the secretion of proresolving mediator 15-HETE and thus reducing inflammation and promoting the resolution of inflammation and thus the return to homeostasis.

In conclusion, the experimental tests have shown that the composition C A according to the invention, comprising the lysate (Ly) of dedifferentiated cells of the plant Helichrysum stoechas, is capable of reducing the sensations of discomfort associated with dry and/or sensitive and/or reactive skin, more particularly of reducing the sensations of itching, stinging, burning, pain and tingling, by stimulating the synthesis of proresolving mediators.

B) Formulations

In the following formulae, the percentages are expressed by weight of the formulation.

B.1 Face Makeup-Removing Fluid

Formula

| | |
|---|---|
| Composition (C_{A}) | 10.00% |
| Methyl paraben | 0.15% |
| Phenoxyethanol | 0.80% |
| Sepicalm™ S | 1.00% |
| Fragrance/Scent | 0.10% |
| Water | q.s. 100.00% |

Procedure: The various ingredients are mixed in the water with magnetic stirring, in the order indicated, and the pH is adjusted to the vicinity of 7.

B.2 Infant Hair and Body Shampoo

Formula

| | | |
|---|---|---|
| A | Composition (C_{A}) | 15.00% |
| | Proteol™ APL | 5.00% |
| | Sepicide™ HB | 0.50% |
| | Fragrance/Scent | 0.10% |
| B | Water | 20.00% |
| | Capigel™ 98 | 3.50% |
| C | Water | q.s. 100.00% |
| | Sepicide™ Cl | 0.30% |
| | Colorant | q.s. |
| | Sodium hydroxide | q.s. pH = 7.2 |

Procedure: The composition (C_{A}) is mixed with the Proteol™ APL and the Sepicide™ HB (Phase A). The Capigel™ 98 is diluted in a portion of the water and is added to phase A obtained above (Phase B). The remainder of the water is added to phase B, followed by the Sepicide™ Cl and the colorant. The pH of the mixture is adjusted to approximately 7.2 with sodium hydroxide.

B.3 Makeup-Removing Wipes for the Eyes

Formula

| | | |
|---|---|---|
| A | Composition (C_{A}) | 3.00% |
| B | Sepicide™ HB2 | 0.50% |
| C | Sepicalm™ VG | 0.50% |
| | Fragrance/Scent | 0.05% |
| D | Water | q.s. 100.00% |

Procedure: The ingredients of phase B and also those of phase C are mixed in phase A until clarity of the solution is obtained. Phase D is added.

B.4 Mild Foaming Gel

Formula

| | | |
|---|---|---|
| A | Composition (C_{A}) | 8.50% |
| | Proteol™ APL | 3.00% |
| | Euxy\|™ PE9010 | 1.00% |
| | Fragrance/Scent | 0.10% |
| B | Water | q.s. 100.00% |
| | Lactic acid | q.s. pH = 6.0 |

Procedure: The fragrance and the preservative Euxyl™ PE9010 are dissolved in the mixture composed of the composition (C_{A}) and the Proteol™ APL (Phase A). The water is added and the pH is adjusted to approximately 6.0 with lactic acid.

B.5 Frequent-Use Shampoo

Formula

| | | |
|---|---|---|
| A | Composition (C_{A}) | 12.80% |
| | Proteol™ OAT | 5.00% |
| | Euxy\|™ PE9010 | 1.00% |
| | Fragrance/Scent | 0.30% |
| | Water q.s. | 100.00% |
| B | Montaline™ C40 | 8.50% |
| | Lactic acid | q.s. pH = 6.0 |

Procedure: All the ingredients of phase A are mixed and, after homogenization, the Montaline™ C40 is added and the pH is adjusted to approximately 6.0 with lactic acid.

B.6 Ultra-Mild Baby Shampoo

Formula

| | | |
|---|---|---|
| A | Composition (C_{A}) | 10.00% |
| | Amisoft™ CS-11 | 4.00% |
| | Fragrance/Scent | 0.10% |
| | Sepicide™ HB | 0.30% |
| | Sepicide™ Cl | 0.20% |
| | Water | q.s. 100.00% |
| B | Water | 20.00% |
| | Capigel™ 98 | 3.50% |
| | Tromethamine | q.s. pH = 7.2 |

Procedure: All the ingredients of phase A are mixed, in the order indicated, until a clear phase A is obtained. Separately, the Capigel™ 98 is added to the water and then this phase B, thus prepared, is added to phase A and the pH is adjusted to 7.2 using tromethamine.

B.7 Baby Cleansing Milk

Formula

| | | |
|---|---|---|
| A | Simulsol™ 165 | 2.00% |
| | Montanov™ 202 | 1.00% |
| | Lanol™ 99 | 3.00% |
| | Dimethicone | 1.00% |
| | Isohexadecane | 3.00% |
| B | Water | q.s. 100.00% |
| C | Sepiplus™ 400 | 0.30% |
| D | Composition (C_{A}) | 6.35% |
| E | Sepicide™ HB | 0.30% |
| | DMDM Hydantoin | 0.20% |
| | Fragrance/Scent | 0.10% |

Procedure: Phases A and B, formed by mixing the various constituents, are heated separately. Phase C is added to the hot fatty phase and the emulsion is produced by pouring in the aqueous phase; homogenization is carried out for a few minutes with vigorous stirring (via a rotor/stator turbomixer). Phase D is then added to the hot emulsion and the emulsion is cooled with moderate stirring until it has returned to ambient temperature. Phase E is added at 40° C.

B.8 Cleansing Powder Lotion for Sensitive Skin

Formula

| | | |
|---|---|---|
| A | Lipacide™ C8G | 0.95% |
| | Methyl paraben | 0.10% |
| | Ethyl paraben | 0.024% |
| | Propyl paraben | 0.0119% |
| | Butyl paraben | 0.024% |
| | Isobutyl paraben | 0.0119% |
| | Water | 20.00% |
| | Disodium EDTA | 0.10% |
| | Triethanolamine | 1.38% |
| B | Composition (C_{A}) | 1.80% |
| | Fragrance/Scent | 0.10% |
| C | Sepicalm™ S | 0.28% |
| | Water | q.s. 100.00% |
| | Lactic acid | q.s. pH = 5.2 |
| D | Micropearl™ M310 | 5.00% |

Procedure: The ingredients of phase A are dissolved in the water at 80° C. Separately, the fragrance is dissolved in the composition (C_{A}) to prepare phase B. The cooled phase A is added to phase B, then the Sepicalm™ S and the remaining water are introduced. The final pH is checked and optionally adjusted to approximately 5.2. The Micropearl™ M310 is then added.

B.9 Kids Shower Gel

Formula

| | | |
|---|---|---|
| A | Water | 56.06% |
| | Sepimax™ Zen | 3.00% |
| | Sepiplus™ S | 0.80% |
| B | Proteol™ OAT | 20.80% |
| | Oramix™ NS 10 | 9.30% |
| | Amony\|™ 265 BA | 5.10% |
| | Composition (C_{A}) | 2.00% |
| | Glyceryl Glucoside | 1.00% |
| | Phenoxyethanol & Ethylhexylglycerin | 1.00% |
| | Fragrance/Scent | 0.90% |
| | Colorant | 0.04% |

Procedure: The Sepimax™ Zen is dispersed in the water and stirred using a mechanical stirrer equipped with a deflocculator, a counter-rotating impeller and a paddle of anchor type, until a perfectly smooth gel is obtained. The Sepiplus™ S is added and then stirring is carried out until the mixture is homogeneous. The ingredients of phase B are subsequently added, the mixture is homogenized and the additives of phase C are added individually. The pH is adjusted to 6.0-6.5.

B.10 BB Cream

Formula

| | | |
|---|---|---|
| A | Easynov™ | 2.30% |
| | Lanol™ 99 | 1.00% |
| | Sepimat™ H10W | 1.00% |
| | Ethylhexyl methoxycinnamate | 5.00% |
| B | Cyclomethicone | 6.00% |
| | Triethoxycaprylsilane & Alumina-Silane & | 8.00% |
| | Titanium Oxide | |
| | Iron Oxide Red & Triethoxycaprylsilane | 0.24% |
| | Iron Oxide Yellow & Triethoxycaprylsilane | 0.66% |
| | Iron Oxide Black & Triethoxycaprylsilane | 0.09% |
| | Fragrance/Scent | 0.10% |
| C | Water | q.s. 100% |
| | Sepinov™ EMT10 | 1.20% |
| D | Composition (CA) | 2.00% |
| | Sepitonic™ M3 | 1.00% |
| | Phenoxyethanol & Ethylhexylglycerin | 1.00% |

Procedure: Phase B is prepared by mixing the various ingredients and the mixture is homogenized using a mixer provided with a rotor-stator system at a rotational speed of 4500 revolutions per minute, for a period of time of 6 minutes. Phase C is prepared by adding the Sepinov™ EMT10 to the mixture of water and glycerol, and the mixture is homogenized using a mixer provided with a rotor-stator system at a rotational speed of 4000 revolutions per minute for 4 minutes. Phases A and B are added to phase C and the resulting mixture is stirred using a mechanical stirrer provided with a paddle of anchor type, at a speed of 30 revolutions per minute for 2 minutes and then at a speed of 50 revolutions per minute for 20 minutes. The components of phase D are added one by one and the mixture is stirred at a speed of 50 revolutions per minute for 25 minutes.

B.11 High-Protection Sun Spray, SPF of Greater than 30

Formula

| | | |
|---|---|---|
| A | Montanov™ L | 1.00% |
| | Montanov™ 82 | 1.00% |
| | C12-15 Alkyl benzoate | 17.00% |
| | Dimethicone | 3.00% |
| | Octocrylene | 6.00% |
| | Ethylhexyl methoxycinnamate | 6.00% |
| | Bis-Ethylhexyloxyphenol Methoxyphenyl Triazine | 3.00% |
| | Tocopherol | 0.05% |
| B | Water | q.s. 100% |
| C | Simulgel™ INS 100 | 0.50% |
| | Cyclodimethicone | 5.00% |
| D | Composition (C_{A}) | 3.00% |
| | Phenoxyethanol & Ethylhexylglycerin | 1.00% |
| | Fragrance/Scent | 0.20% |
| E | Methylene Bis-Benzotriazolyl | 10.00% |
| | Tetramethylbutylphenol | |
| | 25% Citric acid | q.s. pH = 5 |

Sepicalm™ S: Mixture of N-cocoylamino acids, sarcosine, potassium aspartate and magnesium aspartate as described in WO 98/09611;
Proteol™ APL: Mixture of sodium salts of N-cocoylamino acids which are obtained by acylation of the characteristic amino acids of apple juice;
Sepicide™ HB: Mixture of phenoxyethanol, methyl paraben, ethyl paraben, propyl paraben and butyl paraben, which is a preservative;
Capigel™ 98: Copolymer of acrylates;
Sepicide™ CI: Imidazoline urea, which is a preservative;
Sepicide™ HB: Mixture of phenoxyethanol, methyl paraben, ethyl paraben, propyl paraben, butyl paraben and isobutyl paraben, which is a preservative;
Sepicalm™ VG: Mixture of N-palmitoylproline in sodium salt form and of extract of flowers of Nymphea alba;
Euxyl™ PE9010: Mixture of phenoxyethanol and ethylhexylglycerin;
Proteol™ OAT: Mixture of N-lauroylamino acids which are obtained by complete hydrolysis of oat protein, as described in WO 94/26694;
Montaline™ C₄₀: Monoethanolamine cocamidopropyl betainamide chloride salt;
Amisoft™ CS-11: Sodium salt of N-cocoylglutamate;
Simulsol™ 165: Mixture of PEG-100 stearate and glyceryl stearate;
Montanov™ 202 (arachidyl alcohol, behenyl alcohol and arachidyl glucoside) is a self-emulsifiable composition, such as those described in EP 0 977 626;
Lanol™ 99: Isononyl isononanoate;
Sepiplus™ 400: Self-invertible inverse latex of polyacrylates in polyisobutene which comprises polysorbate 20, such as described in WO 2005/040230;
Lipacide™ CBG: Capryloyl glycine, sold by SEPPIC;
Micropearl™ M310: Crosslinked polymethyl methacrylate polymer which is provided in powder form and which is used as texture modifier;
Sepimax™ Zen (INCI name: Polyacrylate Crosspolymer-6): Thickening polymer which is provided in the form of a powder;
Sepiplus™ S (INCI name: Hydroxyethyl Acrylate/Sodium Acryloyldimethyl Taurate Copolymer & Polyisobutene & PEG-7 Trimethylolpropane Coconut Ether): Self-invertible inverse latex;
Amonyl™ 265 BA (INCI name: Cocobetaine): Foaming amphoteric surface-active agent;
Sepinov™ EMT10 (INCI name: Hydroxyethyl Acrylate/Sodium Acryloyldimethyl Taurate Copolymer): Thickening copolymer which is provided in the form of a powder;
Easynov™ (INCI name: Octyldodecanol and Octyldodecyl Xyloside and PEG-30 Dipolyhydroxystearate): Emulsifying agent having a lipophilic tendency;
Sepimat™ H10 FW (INCI name: Methyl Methacrylate Crosspolymer and Squalane): Polymer used as texturing agent;
Sepitonic™ M3 (INCI name: Magnesium Aspartate and Zinc Gluconate and Copper Gluconate): Mixture used as agent for combating free radicals and as energizing ingredient for cells;
Montanov™ L (INCI name: C14-22 Alcohols and C₁₂₋₂₀ Alkylglucoside): Emulsifying agent;
Montanov™ 82 (INCI name: Cetearyl Alcohol and Coco-glucoside): Emulsifying agent;
Simulgel™ INS100 (INCI name: Hydroxyethyl Acrylate/Sodium Acryloyldimethyl Taurate Copolymer and Isohexadecane and Polysorbate 60): Polymeric thickening agent.

1. A lysate of dedifferentiated cells of the plant Helichrysum stoechas in a form suitable for topical administration for eliminating or reducing inflammation of the skin and/or of the scalp.

2. A method of producing the lysate as claimed in claim 1, the method comprising high-pressure homogenization of the culture of dedifferentiated cells of the plant Helichrysum stoechas.

3. A composition suitable for topical use which is provided in the form of a gel and which comprises, per 100% of its weight:
from 95% to 99.5% by weight of the lysate as defined in claim 1,
from 0.5% to 5% by weight of at least one thickening and/or gelling agent.

4. The composition as claimed in claim 3, wherein the gelling and/or thickening agents are chosen from polysaccharides, cellulose and cellulose derivatives, starches and linear or branched or crosslinked polymers of polyelectrolyte type.

5. The composition as claimed in claim 3 in a form suitable for topical administration for reducing or eliminating inflammation of the skin and/or of the scalp.

6. A composition suitable for topical use which is provided in the form of a gel and which comprises, per 100% of its weight:
from 50% to 80% by weight of the lysate as defined in claim 1,
from 0.1% to 5% by weight of at least one thickening and/or gelling agent, and
from 15% to 49.9% by weight of at least one solvent.

7. The composition as claimed in claim 6, wherein the solvent is chosen from the elements of the group consisting of:
the compounds of formula (Ia):
HO—[CH₂—CH(OH)—CH₂—O]—H (Ia)
in which n represents an integer greater than or equal to 1 and less than or equal to 15;
the compounds of formula (Ib):
Ra1-C(Rb1)(OH)—C(OH)(Rc1)(Rd1) (Ib),
in which each of the radicals Ra1, Rb1, Rc1 and Rd1 represent, independently of one another, a hydrogen atom or a saturated aliphatic radical comprising from 1 to 5 carbon atoms, or by the formula (Ib1):
Ra1-C(Rb1)(OH)—[C(Re1)(Rf1)]ₜ-C(OH)(Rc1)(Rd1) (Ib1),
in which t is equal to 1, 2 or 3 and each of the radicals Ra1, Rb1, Rc1, Rd1, Re1 and Rf1 independently represent a hydrogen atom or a saturated aliphatic radical comprising from 1 to 5 carbon atoms, wherein at least one of the Ra1 or Rb1 radicals and/or at least one of the Rc1 or Rd1 radicals does not represent a hydrogen atom.

8. The composition (of claim 6, wherein the solvent is glycerol.

9. The composition of claim 6 in a form suitable for topical administration for reducing or eliminating inflammation of the skin and/or of the scalp.

10. A composition suitable for topical use which is provided in the form of an emulsion of water-in-oil type or of an emulsion of oil-in-water type, comprising the lysate as defined in claim 1.

11. A composition suitable for topical use which is provided in the form of a water-in-oil emulsion and which comprises, per 100% of its weight:
from 60% to 90% by weight of the composition for topical use or as defined in claim 3,
from 10% to 40% by weight of a fatty phase comprising i) at least one oil and ii) an emulsifying system comprising at least one emulsifying surface-active agent.

12. The composition as claimed in claim 11, wherein the emulsifying surface-active agent is chosen from the elements of the group consisting of alkylpolyglycoside compositions, compositions of alkylpolyglycosides and of fatty alcohols, polyglycerol esters, alkoxylated polyglycerol esters, polyglycol polyhydroxystearates, polyglycerol polyhydroxystearates and alkoxylated polyglycerol polyhydroxystearates.

13. A composition suitable for topical use which is provided in the form of an oil-in-water emulsion and which comprises, per 100% of its weight:
from 50% to 90% by weight of a cosmetically acceptable aqueous phase, said aqueous phase comprising, per 100% of its own weight, from 0.5% to 10% by weight of the lysate as defined in claim 1,
from 10% to 50% by weight of a fatty phase comprising, per 100% of its own weight:
from 0.5% to 20% by weight of at least one surface-active agent of oil-in-water type,
from 80% to 99.5% by weight of at least one oil and/or one wax.

14. The composition as claimed in claim 10 in a form suitable for topical administration for reducing or eliminating inflammation of the skin and/or of the scalp.

15. The composition as claimed in claim 10 for reducing and/or eliminating stinging and/or tingling and/or itching and/or heating and/or red patches and/or skin discomfort and/or tightness of the skin caused by inflammation of human skin and/or the scalp.

16. The composition of claim 7, wherein n represents an integer greater than or equal to 1 and less than or equal to 10.

17. The composition of claim 7, wherein n represents an integer greater than or equal to 1 and less than or equal to 6.

18. The composition of claim 7, wherein n represents an integer greater than or equal to 1 and less than or equal to 4.

19. The composition of claim 7, wherein n represents an integer equal to 4.

20. The composition of claim 7, wherein n represents an integer equal to 3.
